(19) United States
(12) Patent Application Publication
(10) Pub. No.: US 2026/0273056 A1
(43) Pub. Date: Sep. 17, 2026

(54) COMPOSITIONS AND METHODS FOR TARGETING SOLID TUMORS WITH CHIMERIC ANTIGEN RECEPTOR (CAR) MACROPHAGES

(71) Applicant: Rutgers, The State University of New Jersey, New Brunswick, NJ (US)

(72) Inventor: Pingping Hou, New Brunswick, NJ (US)

(21) Appl. No.: 19/168,180

(22) PCT Filed: Apr. 9, 2024
(86) PCT No.: PCT/US2024/023741

§ 371 Date: Sep. 23, 2025

Related U.S. Application Data
(60) Provisional application No. 63/496,286, filed on Apr. 14, 2023.

(51) Int. Cl. A61K 40/31; A61K 40/17; A61K 40/40; A61P 35/00; C07K 14/705; C07K 16/44; C12N 5/0786; C12N 15/86

(52) U.S. Cl. CPC A61K 40/31; A61K 40/17; A61K 40/429; A61P 35/00; C07K 14/7051; C07K 16/44; C12N 5/0645; C12N 15/86; C12N 2510/00; C12N 2740/15043

(57) ABSTRACT

Described herein are compositions and methods for treating cancer in a subject. In some embodiments, the compositions and methods may be used for treating solid tumor cancers. In some embodiments, the compositions and methods may comprise a modified macrophage or monocyte comprising a surface-expressed phosphatidylserine (PS)-targeted chimeric antigen receptor (CAR) polypeptide to specifically target solid tumors. The modified macrophage or monocyte comprising the surface-expressed PS-targeted CAR polypeptide may activate an immune response in a tumor microenvironment in the subject, thereby treating the cancer in the subject.

### CROSS-REFERENCE TO RELATED APPLICATIONS

This application is a U.S. national phase application of International Patent Application No. PCT/US2024/023741, filed Apr. 9, 2024, which claims priority to U.S. Provisional Patent Application No. 63/496,286, filed Apr. 14, 2023, each of which is incorporated by reference herein in its entirety.

### FEDERALLY SPONSORED RESEARCH

This invention was made with government support under grant number CA286398 awarded by the National Institutes of Health. The government has certain rights in the invention.

### REFERENCE TO SEQUENCE LISTING

This application was filed with a Sequence Listing XML in ST.26 XML format in accordance with 37 C.F.R. § 1.831. The Sequence Listing XML file submitted in the USPTO Patent Center, “210953-0001-WOO1_sequence_listing_XML_2 Apr. 2024.xml,” was created on Apr. 2, 2024, contains 12 sequences, has a file size of 15.6 Kbytes, and is incorporated by reference in its entirety into the specification.

### FIELD OF THE DISCLOSURE

This disclosure generally relates to compositions and methods for treating cancer in a subject.

### BACKGROUND

Immunotherapies such as immune checkpoint blockade, chimeric antigen receptor (CAR) T cell therapies, and natural killer (NK) cell therapies are promising curative modalities for cancer. However, they have limited efficacy against “cold” solid tumors due to the hostile tumor microenvironment (TME) that prevents lymphocyte infiltration and lacks abundant tumor-specific neoantigens. For example, some types of solid cancers (e.g., pancreatic cancer) have a minimal response to immunotherapy, and one of the major reasons for this is the immunosuppressive TME.

Macrophages (Mφs) are abundant and usually immune-suppressive in solid tumors. Because macrophages are innate immune cells, they are considered allotransplantation permissive. Macrophages serve as phagocytes and professional antigen presenting cells (APCs), which are crucial for tissue homeostasis and adaptive immune response regulation. The high phenotypic and functional plasticity and tumor penetration rate make macrophages ideal for tumor specific delivery as cell therapy donors. To override the immune suppressive gene signatures once infiltrated in solid tumors, macrophages can be engineered with CARs (a type of artificial receptor for cell surface proteins) to redirect the specificity and function of immune cells. However, current CAR-based cell therapies are limited by antigen specificity and scalability, as well as a time-consuming and costly individualized manufacturing process.

Phosphatidylserine (PS) is a negatively-charged amino-phospholipid that is primarily confined to the inner leaflet (i.e., intracellular side) of the plasma membrane bilayer in healthy cells, but PS is flipped to the outer membrane under heavy metabolic or lethal stress, as well as in apoptotic cells. These externalized PS molecules are recognized by macrophages that engulf the distressed cells in a process called efferocytosis. This process leads to the polarization of the macrophage into an anti-inflammatory M2 state. This process serves to allow tissues to undergo wound healing and prevent the triggering of a run-away inflammatory cascade due to high cell death from lesion or trauma.

Interestingly, an abnormally high number of PS molecules are also found to be externalized by cancer cells of solid tumors, despite not undergoing apoptosis. This leads to macrophages efferocytosing tumor cells and polarizing to M2 phenotypes, releasing anti-inflammatory cytokines and promoting wound-healing pathways as well as inhibiting future phagocytic activity. These processes help to protect the tumor by inhibiting tumoricidal immune pathways and such M2 macrophages are often referred to as tumor-associated-macrophages (TAMs).

What is needed are novel therapeutic compositions and methods for specifically targeting the solid-tumor TME to elicit an immune response, prevent therapy resistance, and prolong cancer patient survival. Such compositions and methods would be useful in treating a variety of solid tumor cancers either alone or as an adjuvant cell therapy with other cancer therapeutic modalities. These compositions and methods would also be useful in a variety of clinical, research, and commercial applications.

### SUMMARY

One embodiment described herein is a polynucleotide construct encoding a phosphatidylserine (PS)-targeted chimeric antigen receptor (CAR) polypeptide, the PS-targeted CAR polypeptide comprising: (a) a signal peptide; (b) an extracellular PS-binding domain; (c) a hinge domain; (d) a transmembrane domain; and (e) one or more intracellular immunostimulatory activation domains. In one aspect, the signal peptide comprises an amino acid sequence having at least 90-99% identity to SEQ ID NO: 1 or 6. In another aspect, the extracellular PS-binding domain comprises an amino acid sequence having at least 90-99% identity to one or more portions of SEQ ID NO: 3 or 7. In another aspect, the hinge domain comprises an amino acid sequence having at least 90-99% identity to one or more portions of SEQ ID NO: 3 or 8. In another aspect, the transmembrane domain comprises an amino acid sequence having at least 90-99% identity to one or more portions of SEQ ID NO: 3 or 8. In another aspect, the one or more intracellular immunostimulatory activation domains comprise one or more amino acid sequences having at least 90-99% identity to SEQ ID NO: 4 or 9. In another aspect, the PS-targeted CAR polypeptide comprises an amino acid sequence having at least 90-99% identity to SEQ ID NO: 5 or 10. In another aspect, the PS-targeted CAR polypeptide comprises an amino acid sequence of SEQ ID NO: 5 or 10. In another aspect, the polynucleotide construct comprises a polynucleotide sequence having at least 90-99% identity to SEQ ID NO: 11 or 12. In another aspect, the polynucleotide construct comprises a polynucleotide sequence of SEQ ID NO: 11 or 12.

Another embodiment described herein is a vector comprising any of the polynucleotide constructs described herein, the vector comprising a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof. In one aspect, the vector is a lentiviral vector selected from the group consisting of pHAGE and pLenti.

Another embodiment described herein is a phosphatidylserine (PS)-targeted chimeric antigen receptor (CAR) polypeptide encoded by any of the polynucleotide constructs or vectors described herein.

Another embodiment described herein is a modified macrophage or monocyte comprising any of the polynucleotide constructs, vectors, or PS-targeted CAR polypeptides described herein.

Another embodiment described herein is a modified macrophage or monocyte comprising a surface-expressed phosphatidylserine (PS)-targeted chimeric antigen receptor (CAR) polypeptide, the PS-targeted CAR polypeptide comprising: (a) a signal peptide; (b) an extracellular PS-binding domain; (c) a hinge domain; (d) a transmembrane domain; and (e) one or more intracellular immunostimulatory activation domains. In one aspect, the modified macrophage or monocyte is derived from a primary macrophage or monocyte, or the modified macrophage or monocyte is derived from an induced pluripotent stem cell. In another aspect, the PS-targeted CAR polypeptide comprises an amino acid sequence having at least 90-99% identity to SEQ ID NO: 5 or 10. In another aspect, the PS-targeted CAR polypeptide comprises an amino acid sequence of SEQ ID NO: 5 or 10. In another aspect, the PS-targeted CAR polypeptide is encoded by a polynucleotide construct comprising a polynucleotide sequence having at least 90-99% identity to SEQ ID NO: 11 or 12. In another aspect, the PS-targeted CAR polypeptide is encoded by a polynucleotide construct comprising a polynucleotide sequence of SEQ ID NO: 11 or 12.

Another embodiment described herein is a pharmaceutical composition comprising any of the modified macrophages or monocytes described herein.

Another embodiment described herein is a method of treating cancer in a subject, the method comprising administering to the subject a therapeutically effective amount of a pharmaceutical composition comprising a modified macrophage or monocyte comprising a surface-expressed phosphatidylserine (PS)-targeted chimeric antigen receptor (CAR) polypeptide, the PS-targeted CAR polypeptide comprising: (a) a signal peptide; (b) an extracellular PS-binding domain; (c) a hinge domain; (d) a transmembrane domain; and (e) one or more intracellular immunostimulatory activation domains. In one aspect, the method further comprises administering to the subject a therapeutically effective amount of one or more additional anti-cancer drugs. In another aspect, the one or more additional anti-cancer drugs comprise a KRAS inhibitor, a merTK inhibitor, or a combination thereof. In another aspect, the subject has a solid tumor cancer selected from the group consisting of pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer. In another aspect, the modified macrophage or monocyte comprising the surface-expressed PS-targeted CAR polypeptide activates an immune response in a tumor microenvironment in the subject, thereby treating the cancer in the subject.

This disclosure provides for other aspects and embodiments that will be apparent in light of the following detailed description and accompanying drawings.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-D show the tumoricidal activity of anti-phosphatidylserine (α-PS) CAR-macrophages (CAR-Ms). FIG. 1A shows an illustrative schematic demonstrating the different structural components and the immunostimulatory anti-tumor effect of the disclosed α-PS CAR-Ms. The different structural domains of the α-PS CARs include a signal peptide (SP); an anti-PS extracellular domain (ECD); a hinge domain (HD); a transmembrane domain (TMD); and an intracellular immunostimulatory activation domain specifically optimized for macrophage polarization/activation (ICD). FIG. 1B shows that KRAS inhibition (KRASi) results in PS externalization by Annexin V staining in the human pancreatic ductal adenocarcinoma (PDAC) cell line AsPC-1. FIG. 1C shows that α-PS CAR1-Ms effectively engulf cancer cells having high external expression of PS through phagocytosis. FIG. 1D shows that α-PS CAR2-Ms induce cytotoxicity towards cancer cells having high external expression of PS, and that the cytotoxic activity is enhanced by dual merTK inhibition. A two-tailed t-test was used for statistical analysis.

FIG. 2A-D show further phagocytic activity of α-PS CAR-Ms using flow cytometry analysis in AsPC-1 PDAC cells (FIGS. 2A and 2C) and in mouse PDAC cells derived from KPC (KRASG12D L/+, P53 L/+, p48-Cre+) genetically engineered mouse models (FIGS. 2B and 2D). The phagocytosis % is shown in FIG. 2A-D from flow cytometry analysis of RFP and GFP double positive cells after co-culturing GFP+ anti-PS CAR-Ms and RFP+ PDAC cells with or without KRAS inhibitor treatment. The KRAS G12D inhibitor MRTX1133 was used in this assay. The ratio of the anti-PS CAR-M effector cells to the different targeted cancer cells (E:T ratio) was 1:3 for each experiment. The p-values were analyzed by unpaired two-tailed t-tests for statistical analysis.

FIG. 3A-C show further PS antigen-specific tumor killing activities of α-PS CAR-Ms by cell lysis and bioluminescent analysis in AsPC-1 PDAC cells (FIG. 3A) and in mouse KPC PDAC cells (FIG. 3B-C). The specific lysis % is shown in FIG. 3A-C from bioluminescent analysis of residue luciferase activities in PDAC cells after co-culturing with α-PS CAR-Ms with or without KRAS inhibitor treatment. The KRAS G12D inhibitor MRTX1133 was used in this assay. The ratio of the anti-PS CAR-M effector cells to the different targeted cancer cells (E:T ratio) was 10:1 for the experiments of FIG. 3A-B and 20:1 for the experiment of FIG. 3C. The p-values were analyzed by unpaired two-tailed t-tests for statistical analysis.

FIG. 4A-B show fluorescent images of phagocytosis and autophagy using the IncuCyte® live-cell imaging analysis system. FIG. 4A shows phagocytosis imaging of α-PS CAR2-Ms labeled by GFP (green) and AsPC-1 PDAC cells or mouse KPC PDAC cells labeled by CellTracker® Red (red; red fluorescent protein (RFP)), where the co-staining (yellow) indicates phagocytic events. FIG. 4B shows autophagy activity of α-PS CAR2-Ms labeled by GFP (green), and LysoTracker® Red (red) was used to visualize autophagy activities. The co-staining (yellow) indicates phagocytic events.

Before any embodiments of the disclosure are explained in detail, it is to be understood that the disclosure is not limited in its application to the details of construction and the arrangement of components set forth in the following description or illustrated in the following drawings. Also, it is to be understood that the phraseology and terminology used herein is for the purpose of description and should not be regarded as limiting.

### DETAILED DESCRIPTION

While the disclosure has been described in connection with certain embodiments, it is to be understood that the disclosure is not to be limited to the disclosed embodiments and is intended to cover various modifications and equivalent arrangements included within the scope of the appended claims, which scope is to be accorded the broadest interpretation to encompass all such modifications and equivalent structures as is permitted under the law.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine, hypoxanthine, isocytosine, and isoguanine. Nucleic acids may be obtained by chemical synthesis methods or by recombinant methods.

As used herein, “variants” can include, but are not limited to, those that include conservative amino acid (AA) substitution, SNP variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present disclosure, a CAR polypeptide may be modified, for example, to facilitate or improve activity, stability, identification, expression, isolation, storage, and/or administration, so long as such modifications do not reduce its function to an unacceptable level.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present disclosure encoding a protein or polypeptide of the present disclosure include nucleic acid sequences that have substantial identity to the nucleic acid sequences that encode the proteins or polypeptides of the present disclosure. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present disclosure, include amino acid sequences that have substantial identity to the amino acid sequences of the reference polypeptide sequences.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, cell, or substance, compositions, or mixtures thereof, that provide a pharmacological, therapeutic, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, and/or nanoparticles.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “administering” refers to the placement of an agent, composition, or cell as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions or emulsions. In one embodiment, the agent or composition may be provided in a powder form and mixed with a liquid, such as water, to form a beverage. In accordance with the present disclosure, “administering” can be self-administering. For example, it is considered “administering” when a subject consumes a composition as disclosed herein.

As used herein, “contacting” refers to contacting a target cell (e.g., tumor cell) with an agent (e.g., a modified macrophage or monocyte comprising a surface-expressed PS-targeted CAR polypeptide) using any method that is suitable for placing the agent on, in, or adjacent to the target cell. For example, when the cells are in vitro, contacting the cells with the agent can comprise adding the agent to culture medium containing the cells. For example, when the cells are in vivo, contacting the cells with the agent can comprise administering the agent to a subject.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments. In some embodiments of the present disclosure, a subject is in need of treatment if the subject is suffering from, or at risk of suffering from, one or more types of cancer.

In some embodiments of the present disclosure, a subject may be administered a single dose of the disclosed pharmaceutical agents, compositions, or cells. In other embodiments, the subject may be administered a plurality of doses of the disclosed pharmaceutical compositions over a period of time. For example, in various nonlimiting embodiments, a pharmaceutical composition comprising a modified macrophage or monocyte comprising a surface-expressed PS-targeted CAR polypeptide as described herein may be administered to a subject once a day (SID/QD), twice a day (BID), three times a day (TID), four times a day (QID), or more, so as to administer a therapeutically effective amount of the pharmaceutical composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof. The actual dosing regimen can depend upon many factors, including but not limited to the judgment of a trained physician, the overall condition of the subject, and the specific type of cancer in the subject. The actual dosage can also depend on the determined experimental effectiveness of the specific pharmaceutical composition that is administered. For example, the dosage may be determined based on in vitro responsiveness of relevant cultured cells, or in vivo responses observed in appropriate animal models or human studies.

As used herein, the term “endogenous” refers to any material from or produced inside an organism, cell, tissue, or system.

As used herein, the term “exogenous” refers to any material introduced from or produced outside an organism, cell, tissue, or system.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic manner. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest. In one embodiment of the present disclosure, a method of treating, preventing, reducing the likelihood of having, reducing the severity of, and/or slowing the progression of cancer in a subject is described.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, “target analyte,” “target biomarker,” “target antigen,” or “target cell” refers to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, a tumor cell, etc.), small molecules, lipids, carbohydrates, nucleic acids, peptides, proteins, enzymes, antigens, and antibodies. A biomarker can be derived from an infectious agent, such as a bacterium, fungus, or virus, or can be an endogenous molecule that is found in greater or lesser abundance in a subject suffering from a disease or disorder as compared to a healthy individual (e.g., an increase or decrease in expression of a gene or gene product).

As used herein, the term “cancer” refers to a disease characterized by the rapid and uncontrolled growth of aberrant cells. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal or kidney cancer, liver cancer, brain cancer, neck cancer, stomach cancer, esophageal cancer, lymphoma, blood cancer, leukemia, myeloma, lung cancer, and the like. As used herein, “solid tumor cancer” refers to sarcomas, carcinomas, and lymphomas where one or more abnormal tissue masses (i.e., tumors) are formed. “Solid tumor cancer” may also refer to a “non-blood cancer,” as commonly known in the art. In certain embodiments of the present disclosure, solid tumor cancer may comprise pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, or skin cancer. In certain embodiments of the present disclosure, a subject may be treated for a solid tumor cancer.

As used herein, the term “anti-tumor effect” refers to a biological effect which can be manifested by a decrease in tumor volume, a decrease in the number of tumor cells, a decrease in activity of pro-tumor or oncogenic molecular signaling transduction, an increase in activity of tumor suppressive molecular signaling transduction, an increase in activity of pro-immune or pro-inflammatory molecular signaling transduction, a decrease in the number of metastases, an increase in life expectancy, amelioration of various physiological symptoms associated with the cancerous condition, and the like. An “anti-tumor effect” can also be manifested by the ability of polypeptides, polynucleotides, cells, and pharmaceutical compositions of the present disclosure in prevention of the occurrence of a tumor in the first place.

As used herein, the term “cytotoxic” or “cytotoxicity” refers to killing or damaging cells. In one embodiment, cytotoxicity of monocyte or macrophage cells is improved (e.g., increased cytolytic or phagocytic activity of macrophages).

As used herein, the term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses (i.e., lentiviral vectors) offer the means to achieve significant levels of gene transfer in vivo. In certain non-limiting exemplary embodiments of the present disclosure, a lentiviral expression vector selected from pHAGE or pLenti may be used to modify an immune cell to express a CAR.

As used herein, the term “immune effector cell” refers to a macrophage or a monocyte. Monocytes and macrophages are similar types of immune cells that are members of the mononuclear phagocyte system, which is a component of innate immunity.

As used herein, the term “modified” refers to a changed state or structure of a molecule or cell of the present disclosure. Molecules may be modified in many ways, including chemically, structurally, and functionally. Cells may be modified through the introduction of nucleic acids or proteins. For example, in some embodiments, a modified macrophage or monocyte is described that comprises a surface-expressed PS-targeted CAR polypeptide, or that comprises a polynucleotide construct or vector encoding such PS-targeted CAR polypeptides.

As used herein, the term “immune response” is defined as a cellular response to an antigen that occurs when an immune effector cell identifies antigenic molecules as foreign and induces the formation of antibodies and/or activates immune effector cells to remove the antigen. In some embodiments, the immune response may comprise the polarization of a macrophage (transition from an M2 anti-inflammatory macrophage to an M1 pro-inflammatory macrophage), mimicking the activation of the IgG-dependent phagocytosis pathway, activation of which elevates phagocytosis and promotes the secretion of pro-inflammatory cytokines such as TNFα and type I interferons (IFNs). This immune response can then lead to phagocytosis and killing of antigen-expressing target cells.

As used herein, the term “chimeric antigen receptor” or “CAR” refers to an artificial cell surface receptor that is engineered to be expressed on an immune effector cell and specifically bind to an antigen. CARs may be used as a therapy with adoptive cell transfer. The structure of CAR constructs may be modulated based on the intended target antigen and the specific immune cell type comprising the CAR. Immune effector cells such as monocytes or macrophages may be removed from a patient (blood, tumor, or ascites fluid) and modified (e.g., using a lentiviral vector expression system) so that they express the CARs specific to a particular form of antigen. In some embodiments, a CAR may target cancers by redirecting a monocyte or macrophage expressing the CAR specific for tumor associated antigens. In some embodiments, the CARs have been expressed with specificity to a tumor-associated antigen. In certain embodiments, the tumor-associated antigen is phosphatidylserine (PS) expressed on the surface of a tumor cell.

The disclosed CARs have a modular design and typically comprise five major components: a signal peptide (SP) to direct the engineered CAR protein into the endoplasmic reticulum and onto the cell surface; an extracellular antigen-binding domain (ECD) to recognize a target antigen and redirect the specificity of CAR-expressing cells; a hinge domain (HD) to provide sufficient flexibility for accessing the antigen; a transmembrane domain (TMD) to anchor and stabilize the CAR on the cell membrane; and an intracellular signal domain (ICD) to stimulate signal transduction in the immune effector cell. Optimal molecular design of CAR polypeptide constructs can be achieved through many variations of these constituent protein domains. The CAR polypeptide may be modified for better activity, stability, expression, production, storage, administration, detection, delivery efficiency, etc. For example, in one non-limiting exemplary embodiment, the CAR polypeptide may be modified with one or more molecular tags and/or linkers such as an HA tag for detection of CARs on cell membranes.

As used herein, the term “signal peptide” refers to an amino acid sequence of a CAR polypeptide that directs the engineered CAR polypeptide first into the endoplasmic reticulum of a cell and then to the cell surface for plasma membrane localization (i.e., surface-expression of the CAR polypeptide). In certain non-limiting exemplary embodiments of the present disclosure, a signal peptide may comprise the signal peptide from the protein T-cell immunoglobulin mucin receptor 1 (TIM1; UniProt ID: Q96D42) or the signal peptide from the protein T-cell surface glycoprotein CD8 alpha chain (CD8A; UniProt ID: P01732).

As used herein, the term “extracellular phosphatidylserine-binding domain” or “extracellular PS-binding domain” refers to an antigen-binding domain of a CAR polypeptide that is specific to a phosphatidylserine molecule. The extracellular PS-binding domain comprises an amino acid sequence having specific binding affinity to one or more regions of a phosphatidylserine molecule. In certain non-limiting exemplary embodiments of the present disclosure, an extracellular PS-binding domain may comprise the extracellular domain from the TIM1 protein. In other non-limiting exemplary embodiments of the present disclosure, an extracellular PS-binding domain may comprise the variable light chain and heavy chain from an anti-PS antibody clone 11.31 as described in US2011/0318360A1, which is hereby incorporated by reference in its entirety into the present specification. For example, the extracellular PS-binding domain may comprise a single-chain variable fragment (scFv) comprising the variable light chain and heavy chain from the anti-PS antibody clone 11.31. Upon binding of the extracellular PS-binding domain to a phosphatidylserine molecule on a tumor cell, a conformational change occurs in the CAR polypeptide which can then activate a pro-immune response from an immune effector cell comprising the CAR polypeptide. For example, a macrophage expressing the CAR polypeptide can become polarized and promote the release of pro-inflammatory cytokines to have an anti-tumor effect on the tumor cell.

As used herein, the term “hinge domain” refers to an amino acid sequence of a CAR polypeptide that has sufficient flexibility to allow access and close proximity of the CAR polypeptide and immune effector cell to a target cell or antigen. In certain non-limiting exemplary embodiments of the present disclosure, a hinge domain may comprise the hinge domain from the TIM1 protein. In other non-limiting exemplary embodiments of the present disclosure, a hinge domain may comprise the hinge domain from the protein T-cell surface glycoprotein CD8 alpha chain (CD8A; UniProt ID: P01732).

As used herein, the term “transmembrane domain” refers to an amino acid sequence of a CAR polypeptide that traverses the plasma membrane of an immune cell and helps to anchor the CAR polypeptide on the plasma membrane for stable surface expression and provides a functional signaling link between the extracellular and intracellular regions of the CAR. In certain non-limiting exemplary embodiments of the present disclosure, a transmembrane domain may comprise the transmembrane domain from the TIM1 protein. In other non-limiting exemplary embodiments of the present disclosure, a transmembrane domain may comprise the transmembrane domain from the CD8A protein.

As used herein, the term “intracellular immunostimulatory activation domain” refers to an amino acid sequence of a CAR polypeptide that stimulates signal transduction in an immune effector cell upon binding of the CAR polypeptide to an antigen, thereby activating an immune response. For example, a signal transduction pathway may be activated in an immune effector cell by a conformational change in the intracellular immunostimulatory activation domain to stimulate a pro-inflammatory immune response where cytokines are released from the immune effector cell, thereby inducing phagocytosis and cytotoxicity of a target tumor cell. In some embodiments, the intracellular immunostimulatory activation domain may stimulate pro-inflammatory macrophage polarization. In certain non-limiting exemplary embodiments of the present disclosure, an intracellular immunostimulatory activation domain may comprise a modified intracellular domain from the protein T-cell surface glycoprotein CD3 zeta chain (CD3Z; UniProt ID: P20963) where residues 54, 88, 99, 104, 116, 118, 129, 136, and 150 are mutated from a lysine (K) to an arginine (R). These mutations alter specific ubiquitination sites on the CD3Z intracellular domain and prevent ubiquitin-dependent CAR degradation to enhance the overall stability and activity of the disclosed CAR constructs. In other non-limiting exemplary embodiments of the present disclosure, an intracellular immunostimulatory activation domain may comprise the intracellular domain from the protein tumor necrosis factor receptor superfamily member 9 (TNFRSF9; UniProt ID: Q07011). In other non-limiting exemplary embodiments of the present disclosure, an intracellular immunostimulatory activation domain may comprise both a modified intracellular domain from the CD3Z protein and the intracellular domain from the TNFRSF9 protein.

When the disclosed anti-PS CAR polypeptide constructs interact with PS externalized on a tumor cell surface, the conformation of the CAR is changed, leading to phosphorylation of immunoreceptor tyrosine-based activation motifs (ITAMs) on the intracellular immunostimulatory activation domains (e.g., CD3Z and/or TNFRSF9) and subsequent activation of signal transduction. Fc receptor γ chain (FcRγ) is a canonical signaling molecule for antibody-dependent cellular phagocytosis in macrophages, which has homology to CD3Z and possesses only one ITAM. Both intracellular domains of FcRγ and CD3Z have the ability to activate CAR-Ms. The clustering of activated FcγRIII results in the phosphorylation of ITAMs in an associated FcRγ dimer, which interacts with FcγRIII via a transmembrane domain. An increased number of ITAMs is known to enhance CAR-T cell activity. Therefore, the intracellular domain of CD3Z may be used over that of FcRγ because CD3Z has three ITAMs.

The disclosed PS-targeted CAR macrophage cell therapies (α-PS CAR-Ms) provide a potential “off-the-shelf” universal adjuvant “helper” therapy that may be used in conjunction with traditional cancer treatment approaches. The disclosed compositions and methods exploit the “tumor-homing” and “immune-modulating” hallmarks of innate immune cell type macrophages and the sole expression of PS on dying apoptotic cells and malignant cells to provoke tumoricidal immune response in solid tumors. This unique immunotherapy approach has the potential to transform care, improve patient outcomes, and save millions of patient lives. Solid tumor malignancies such as advanced lung, colon, and pancreatic cancers are lethal, and patients desperately need innovative and effective therapeutics to prolong survival.

The disclosed α-PS CAR-M therapy has the potential to penetrate solid tumors to provoke tumoricidal immune response. This disclosed technology may be used alone for cancer treatment, or may provide a robust adjuvant therapeutic regimen for synergistic cancer treatment. This technology also provides broader therapeutic value than current CAR-T or NK cell therapies because macrophages exhibit high solid tumor penetration, phagocytic activity, and pro-inflammatory potential, making them superior to T cells and NK cells. Moreover, macrophages are APCs that can also engage T cells to boost tumoricidal immune response. In addition, the present disclosure explores optimal CAR constructs to specifically augment macrophage tumoricidal activities by employing their unique activation and co-stimulatory signaling pathways rather than T cells. Two key characteristics of the present disclosure are the potential to be “off-the-shelf” and non-specific to patients, allowing for greater cost-effective use, as well as the ability to promote the adoption of a pro-inflammatory M1 state in macrophages and downregulate an M2 anti-inflammatory phenotype that is common in cancer-associated monocytes.

Another important distinguishing feature of the present disclosure is the use of CAR-Ms derived from induced pluripotent stem cells (iPSCs). Most CAR cell platforms are derived from genetically modified hematopoietic or progenitor blood cell populations from specific patients, making them personalized and constrained to use on only one person. This makes the therapy very expensive, as economies of scale are difficult to employ, and large-scale standardization of manufacturing and product quality cannot be leveraged to reduce costs. It also greatly increases the implementation time as cells must be harvested from patients, transported to state-of-the-art facilities, transduced over the course of several weeks, purified, and then shipped back to the patient for infusion. Infusions of such concentrated volumes of lymphocytes also have harsh side effects, as the majority of patients receiving these treatments experience serious and sometimes life-threatening immunological complications such as Cytokine Release Syndrome, and Immune Effector Cell-Associated Neurotoxicity. The technological profile of the disclosed compositions and methods avoids many of these issues.

The use of iPSCs as the source population for the disclosed CAR-M cells provides two main benefits. First, iPSCs are allogenic and not patient-derived, meaning that they can be a generalized therapy that can be standardized and used among many patients of differing demographics and genetic profiles. Second, the use of iPSCs provides a path to an optimized, off-the-shelf product profile that can be stored and manufactured at scale. The lack of need for personalized production means that CAR-M cells can be produced and frozen in advance, long before a patient even begins primary lines of therapy for their cancer. Such a system allows for broad standardization and scaling across the manufacturing pipeline, greatly reducing long term cost as well as making treatment more accessible to patients as savings in production are reflected at the consumer/patient level. Macrophages themselves are believed to elicit a minimal HLA-driven incompatibility response, and also do not promote as strong of a cytokine response and aberrant immunological response as seen in CAR-T treated patients.

One embodiment described herein is a polynucleotide construct encoding a phosphatidylserine (PS)-targeted chimeric antigen receptor (CAR) polypeptide, the PS-targeted CAR polypeptide may comprise: (a) a signal peptide; (b) an extracellular PS-binding domain; (c) a hinge domain; (d) a transmembrane domain; and (e) one or more intracellular immunostimulatory activation domains. In one aspect, the signal peptide may comprise an amino acid sequence having at least 90-99% identity to SEQ ID NO: 1 or 6. In another aspect, the extracellular PS-binding domain may comprise an amino acid sequence having at least 90-99% identity to one or more portions of SEQ ID NO: 3 or 7. In another aspect, the hinge domain may comprise an amino acid sequence having at least 90-99% identity to one or more portions of SEQ ID NO: 3 or 8. In another aspect, the transmembrane domain may comprise an amino acid sequence having at least 90-99% identity to one or more portions of SEQ ID NO: 3 or 8. In another aspect, the one or more intracellular immunostimulatory activation domains may comprise one or more amino acid sequences having at least 90-99% identity to SEQ ID NO: 4 or 9. In another aspect, the PS-targeted CAR polypeptide may comprise an amino acid sequence having at least 90-99% identity to SEQ ID NO: 5 or 10. In another aspect, the PS-targeted CAR polypeptide may comprise an amino acid sequence of SEQ ID NO: 5 or 10. In another aspect, the polynucleotide construct may comprise a polynucleotide sequence having at least 90-99% identity to SEQ ID NO: 11 or 12. In another aspect, the polynucleotide construct may comprise a polynucleotide sequence of SEQ ID NO: 11 or 12.

Another embodiment described herein is a vector comprising any of the polynucleotide constructs described herein, the vector may comprise a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof. In one aspect, the vector may be a lentiviral vector selected from the group consisting of pHAGE and pLenti backbones.

Another embodiment described herein is a phosphatidylserine (PS)-targeted chimeric antigen receptor (CAR) polypeptide encoded by any of the polynucleotide constructs or vectors described herein.

Another embodiment described herein is a modified macrophage or monocyte comprising any of the polynucleotide constructs, vectors, or PS-targeted CAR polypeptides described herein.

Another embodiment described herein is a modified macrophage or monocyte comprising a surface-expressed phosphatidylserine (PS)-targeted chimeric antigen receptor (CAR) polypeptide, the PS-targeted CAR polypeptide may comprise: (a) a signal peptide; (b) an extracellular PS-binding domain; (c) a hinge domain; (d) a transmembrane domain; and (e) one or more intracellular immunostimulatory activation domains. In one aspect, the modified macrophage or monocyte may be derived from a primary macrophage or monocyte, or the modified macrophage or monocyte may be derived from an induced pluripotent stem cell. In another aspect, the PS-targeted CAR polypeptide may comprise an amino acid sequence having at least 90-99% identity to SEQ ID NO: 5 or 10. In another aspect, the PS-targeted CAR polypeptide may comprise an amino acid sequence of SEQ ID NO: 5 or 10. In another aspect, the PS-targeted CAR polypeptide may be encoded by a polynucleotide construct comprising a polynucleotide sequence having at least 90-99% identity to SEQ ID NO: 11 or 12. In another aspect, the PS-targeted CAR polypeptide may be encoded by a polynucleotide construct comprising a polynucleotide sequence of SEQ ID NO: 11 or 12.

Another embodiment described herein is a pharmaceutical composition comprising any of the modified macrophages or monocytes described herein.

Another embodiment described herein is a method of treating cancer in a subject, the method may comprise administering to the subject a therapeutically effective amount of a pharmaceutical composition comprising a modified macrophage or monocyte comprising a surface-expressed phosphatidylserine (PS)-targeted chimeric antigen receptor (CAR) polypeptide, the PS-targeted CAR polypeptide may comprise: (a) a signal peptide; (b) an extracellular PS-binding domain; (c) a hinge domain; (d) a transmembrane domain; and (e) one or more intracellular immunostimulatory activation domains. In one aspect, the method may further comprise administering to the subject a therapeutically effective amount of one or more additional anti-cancer drugs. In another aspect, the one or more additional anti-cancer drugs may comprise a KRAS inhibitor, a merTK inhibitor, or a combination thereof. In another aspect, the subject may have a solid tumor cancer selected from the group consisting of pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer. In another aspect, the modified macrophage or monocyte comprising the surface-expressed PS-targeted CAR polypeptide may activate an immune response in a tumor microenvironment in the subject, thereby treating the cancer in the subject.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A polynucleotide construct encoding a phosphatidylserine (PS)-targeted chimeric antigen receptor (CAR) polypeptide, the PS-targeted CAR polypeptide comprising:
   (a) a signal peptide;
   (b) an extracellular PS-binding domain;
   (c) a hinge domain;
   (d) a transmembrane domain; and
   (e) one or more intracellular immunostimulatory activation domains.
Clause 2. The polynucleotide construct of clause 1, wherein the signal peptide comprises an amino acid sequence having at least 90-99% identity to SEQ ID NO: 1 or 6.
Clause 3. The polynucleotide construct of clause 1 or 2, wherein the extracellular PS-binding domain comprises an amino acid sequence having at least 90-99% identity to one or more portions of SEQ ID NO: 3 or 7.
Clause 4. The polynucleotide construct of any one of clauses 1-3, wherein the hinge domain comprises an amino acid sequence having at least 90-99% identity to one or more portions of SEQ ID NO: 3 or 8.
Clause 5. The polynucleotide construct of any one of clauses 1-4, wherein the transmembrane domain comprises an amino acid sequence having at least 90-99% identity to one or more portions of SEQ ID NO: 3 or 8.
Clause 6. The polynucleotide construct of any one of clauses 1-5, wherein the one or more intracellular immunostimulatory activation domains comprise one or more amino acid sequences having at least 90-99% identity to SEQ ID NO: 4 or 9.
Clause 7. The polynucleotide construct of any one of clauses 1-6, wherein the PS-targeted CAR polypeptide comprises an amino acid sequence having at least 90-99% identity to SEQ ID NO: 5 or 10.
Clause 8. The polynucleotide construct of any one of clauses 1-7, wherein the PS-targeted CAR polypeptide comprises an amino acid sequence of SEQ ID NO: 5 or 10.
Clause 9. The polynucleotide construct of any one of clauses 1-8, wherein the polynucleotide construct comprises a polynucleotide sequence having at least 90-99% identity to SEQ ID NO: 11 or 12.
Clause 10. The polynucleotide construct of any one of clauses 1-9, wherein the polynucleotide construct comprises a polynucleotide sequence of SEQ ID NO: 11 or 12.
Clause 11. A vector comprising the polynucleotide construct of any one of clauses 1-10, the vector comprising a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof.
Clause 12. The vector of clause 11, wherein the vector is a lentiviral vector selected from the group consisting of pHAGE and pLenti.
Clause 13. A phosphatidylserine (PS)-targeted chimeric antigen receptor (CAR) polypeptide encoded by the polynucleotide construct or vector of any one of clauses 1-12.
Clause 14. A modified macrophage or monocyte comprising the polynucleotide construct, vector, or PS-targeted CAR polypeptide of any one of clauses 1-13.
Clause 15. A modified macrophage or monocyte comprising a surface-expressed phosphatidylserine (PS)-targeted chimeric antigen receptor (CAR) polypeptide, the PS-targeted CAR polypeptide comprising:
   (a) a signal peptide;
   (b) an extracellular PS-binding domain;
   (c) a hinge domain;
   (d) a transmembrane domain; and
   (e) one or more intracellular immunostimulatory activation domains.
Clause 16. The modified macrophage or monocyte of clause 15, wherein the modified macrophage or monocyte is derived from a primary macrophage or monocyte, or wherein the modified macrophage or monocyte is derived from an induced pluripotent stem cell.
Clause 17. The modified macrophage or monocyte of clause 15 or 16, wherein the PS-targeted CAR polypeptide comprises an amino acid sequence having at least 90-99% identity to SEQ ID NO: 5 or 10.
Clause 18. The modified macrophage or monocyte of any one of clauses 15-17, wherein the PS-targeted CAR polypeptide comprises an amino acid sequence of SEQ ID NO: 5 or 10.
Clause 19. The modified macrophage or monocyte of any one of clauses 15-18, wherein the PS-targeted CAR polypeptide is encoded by a polynucleotide construct comprising a polynucleotide sequence having at least 90-99% identity to SEQ ID NO: 11 or 12.
Clause 20. The modified macrophage or monocyte of any one of clauses 15-19, wherein the PS-targeted CAR polypeptide is encoded by a polynucleotide construct comprising a polynucleotide sequence of SEQ ID NO: 11 or 12.
Clause 21. A pharmaceutical composition comprising the modified macrophage or monocyte of any one of clauses 15-20.
Clause 22. A method of treating cancer in a subject, the method comprising administering to the subject a therapeutically effective amount of a pharmaceutical composition comprising a modified macrophage or monocyte comprising a surface-expressed phosphatidylserine (PS)-targeted chimeric antigen receptor (CAR) polypeptide, the PS-targeted CAR polypeptide comprising:
   (a) a signal peptide;
   (b) an extracellular PS-binding domain;
   (c) a hinge domain;
   (d) a transmembrane domain; and
   (e) one or more intracellular immunostimulatory activation domains.
Clause 23. The method of clause 22, further comprising administering to the subject a therapeutically effective amount of one or more additional anti-cancer drugs.
Clause 24. The method of clause 22 or 23, wherein the one or more additional anti-cancer drugs comprise a KRAS inhibitor, a merTK inhibitor, or a combination thereof.
Clause 25. The method of any one of clauses 22-24, wherein the subject has a solid tumor cancer selected from the group consisting of pancreatic cancer, breast cancer, lung cancer, brain cancer, neck cancer, ovarian cancer, cervical cancer, prostate cancer, kidney cancer, liver cancer, colorectal cancer, stomach cancer, esophageal cancer, and skin cancer.
Clause 26. The method of any one of clauses 22-25, wherein the modified macrophage or monocyte comprising the surface-expressed PS-targeted CAR polypeptide activates an immune response in a tumor microenvironment in the subject, thereby treating the cancer in the subject.

### EXAMPLES

### Example 1

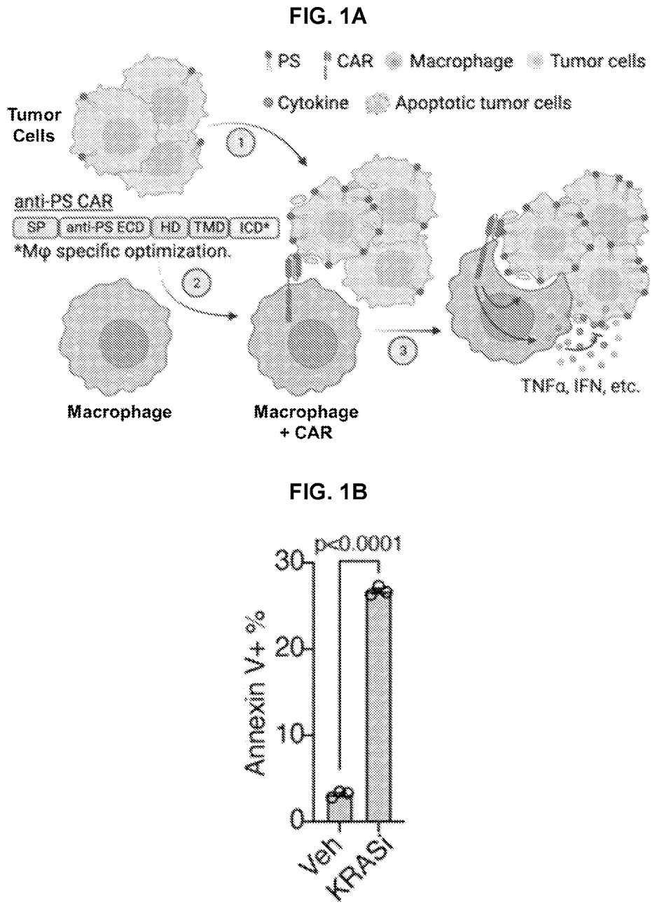

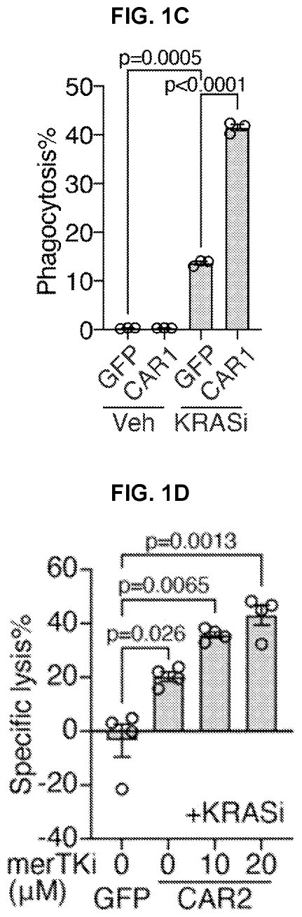

Design of anti-phosphatidylserine (α-PS) chimeric antigen receptors (CARs) An illustrative schematic showing the general structural features and the anti-tumor effect of the disclosed α-PS CAR-Ms is shown in FIG. 1A. An initial construct of an α-PS CAR (i.e., α-PS CAR1) was developed that comprises the following sequence elements: the signal peptide from the protein Hepatitis A virus cellular receptor 1/T-cell immunoglobulin mucin receptor 1 (HAVCR1/TIM1; UniProt ID: Q96D42; amino acids 1-20), which facilitates CAR localization on the cell membrane, shown below as SEQ ID NO: 1 in Table 1; a human influenza hemagglutinin (HA) tag, which is used for detection of CAR expression on the cell membrane, shown below as SEQ ID NO: 2 in Table 1; the extracellular domain, hinge domain, and transmembrane domain from the HAVCR1/TIM1 protein (UniProt ID: Q96D42; amino acids 21-316), which recognize and bind PS and maintain intact CAR structure, shown below as SEQ ID NO: 3 in Table 1; and a modified intracellular domain from the protein T-cell surface glycoprotein CD3 zeta chain (CD3Z; UniProt ID: P20963; amino acids 52-164) having residues 54, 88, 99, 104, 116, 118, 129, 136, and 150 mutated from a lysine to an arginine, which activates an antibody-dependent phagocytosis signaling pathway and enhances CAR activity and stability, shown below as SEQ ID NO: 4 in Table 1. These mutations prevent ubiquitin-dependent CAR degradation so that CAR activity and stability are enhanced. The full α-PS CAR1 protein sequence (438 amino acids long) comprising each of these combined sequence elements is shown below as SEQ ID NO: 5 in Table 1.

TABLE 1
α-PS CAR1 Polypeptide Sequence Elements

| Sequence | | SEQ |
|---|---|---|
| Element (length) | Amino Acid Sequence | ID NO |
| TIM1 signal | MHPQVVILSLILHLADSVAG | 1 |
| peptide (20 aa) | | |
| HA tag (9 aa) | YPYDVPDYA | 2 |
| TIM1 extracellular, | SVKVGGEAGPSVTLPCHYSGAVTSMCWNRGSCSLFTCQNGIVWTNGTHVTY | 3 |
| hinge, and | RKDTRYKLLGDLSRRDVSLTIENTAVSDSGVYCCRVEHRGWFNDMKITVSL | |
| transmembrane | EIVPPKVTTTPIVTTVPTVTTVRTSTTVPTTTTVPMTTVPTTTVPTTMSIP | |
| domains (296 aa) | TTTTVLTTMTVSTTTSVPTTTSIPTTTSVPVTTTVSTFVPPMPLPRQNHEP | |
| | VATSPSSPQPAETHPTTLQGAIRREPTSSPLYSYTTDGNDTVTESSDGLWN | |
| | NNQTQLFLEHSLLTANTTKGIYAGVCISVLVLLALLGVIIA | |
| Modified CD3Z | RVRFSRSADAPAYQQGQNQLYNELNLGRREEYDVLDRRRGRDPEMGGRPQR | 4 |
| intracellular | RRNPQEGLYNELQRDRMAEAYSEIGMRGERRRGRGHDGLYQGLSTATRDTY | |
| domain (K→R | DALHMQALPPR | |
| mutations shown | | |
| as R) (113 aa) | | |
| Full α-PS CAR1 | MHPQVVILSLILHLADSVAGYPYDVPDYASVKVGGEAGPSVTLPCHYSGAV | |
| protein sequence | TSMCWNRGSCSLFTCQNGIVWTNGTHVTYRKDTRYKLLGDLSRRDVSLTIE | |
| (438 aa) | NTAVSDSGVYCCRVEHRGWFNDMKITVSLEIVPPKVTTTPIVTTVPTVTTV | |
| | RTSTTVPTTTTVPMTTVPTTTVPTTMSIPTTTTVLTTMTVSTTTSVPTTTS | |
| | IPTTTSVPVTTTVSTFVPPMPLPRQNHEPVATSPSSPQPAETHPTTLQGAI | 5 |
| | RREPTSSPLYSYTTDGNDTVTESSDGLWNNNQTQLFLEHSLLTANTTKGIY | |
| | AGVCISVLVLLALLGVIIARVRFSRSADAPAYQQGQNQLYNELNLGRREEY | |
| | DVLDRRRGRDPEMGGRPQRRRNPQEGLYNELQRDRMAEAYSEIGMRGERRR | |
| | GRGHDGLYQGLSTATRDTYDALHMQALPPR | |

A second α-PS CAR construct was developed (i.e., α-PS CAR2) that comprises the following sequence elements: the signal peptide from the protein T-cell surface glycoprotein CD8 alpha chain (CD8A; UniProt ID: P01732; amino acids 1-21), which facilitates CAR localization on the cell membrane, shown below as SEQ ID NO: 6 in Table 2; an HA tag, which is used for detection of CAR expression on the cell membrane, shown below as SEQ ID NO: 2 in Table 2; the variable light chain and heavy chain from the anti-PS antibody clone 11.31, which recognizes and binds PS, shown below as SEQ ID NO: 7 in Table 2 and described in US2011/0318360A1; the hinge domain and the transmembrane domain from the CD8A protein (UniProt ID: P01732; amino acids 138-182), which maintain CAR construct stability and mediate CAR dimerization to enhance signaling transduction, shown below as SEQ ID NO: 8 in Table 2; the intracellular domain from the protein tumor necrosis factor receptor superfamily member 9 (TNFRSF9; UniProt ID: Q07011; amino acids 214-255), which enhances macrophage cytotoxicity and acts as a co-stimulatory domain, shown below as SEQ ID NO: 9 in Table 2; and the modified intracellular domain from the CD3Z protein (UniProt ID: P20963; amino acids 52-164) having residues 54, 88, 99, 104, 116, 118, 129, 136, and 150 mutated from a lysine to an arginine, which activates an antibody-dependent phagocytosis signaling pathway and enhances CAR activity and stability, shown below as SEQ ID NO: 4 in Table 2. These mutations prevent ubiquitin-dependent CAR degradation so that CAR activity and stability are enhanced. The full α-PS CAR2 protein sequence (507 amino acids long) comprising each of these combined sequence elements is shown below as SEQ ID NO: 10 in Table 2.

TABLE 2
α-PS CAR2 Polypeptide Sequence Elements

| Sequence | | SEQ |
|---|---|---|
| Element (length) | Amino Acid Sequence | ID NO |
| CD8A signal | MALPVTALLLPLALLLHAARP | 6 |
| peptide (21 aa) | | |
| HA tag (9 aa) | YPYDVPDYA | 2 |
| Variable light and | SSELSQDPAVSVALGQTVRITCQGDSLRSYYASWYQQKPGQAPVLVIYGK | 7 |
| heavy chain | NNRPSGIPDRFSGSSSGNTASLTITGAQAEDEADYYCNSRDSSGNVVFGG | |
| regions from α-PS | GTKVTVLGGGGSGGGGSGGGGSGGGGSQVQLQESGPGLVKPSGTLSLTCA | |
| antibody clone | VSGGSISSSNWWSWVRQPPGKGLEWIGEIYHSGSTNYNPSLKSRVTISVD | |
| | KSKNQFSLKLSSVTAADTAVYYCARSRFRSWLVKRRYVYFDYWGQGTLVT | |
| 11.31* (277 aa) | VSSTTTPAPRPPTPAPTIASQPLSLRP | |
| CD8A hinge and | EACRPAAGGAVHTRGLDFACDIYIWAPLAGTCGVLLLSLVITLYC | 8 |
| transmembrane | | |
| domains (45 aa) | | |
| TNFRSF9 | KRGRKKLLYIFKQPFMRPVQTTQEEDGCSCRFPEEEEGGCEL | 9 |
| intracellular domain | | |
| (42 aa) | | |
| Modified CD3Z | RVRFSRSADAPAYQQGQNQLYNELNLGRREEYDVLDRRRGRDPEMGGRPQ | 4 |
| intracellular domain | RRRNPQEGLYNELQRDRMAEAYSEIGMRGERRRGRGHDGLYQGLSTATRD | |
| (K→R mutations | TYDALHMQALPPR | |
| shown as R) (113 aa) | | |
| Full a-PS CAR2 | MALPVTALLLPLALLLHAARPYPYDVPDYASSELSQDPAVSVALGQTVRI | 10 |
| protein sequence | TCQGDSLRSYYASWYQQKPGQAPVLVIYGKNNRPSGIPDRFSGSSSGNTA | |
| (507 aa) | SLTITGAQAEDEADYYCNSRDSSGNVVFGGGTKVTVLGGGGSGGGGSGGG | |
| | GSGGGGSQVQLQESGPGLVKPSGTLSLTCAVSGGSISSSNWWSWVRQPPG | |
| | KGLEWIGEIYHSGSTNYNPSLKSRVTISVDKSKNQFSLKLSSVTAADTAV | |
| | YYCARSRFRSWLVKRRYVYFDYWGQGTLVTVSSTTTPAPRPPTPAPTIAS | |
| | QPLSLRPEACRPAAGGAVHTRGLDFACDIYIWAPLAGTCGVLLLSLVITL | |
| | YCKRGRKKLLYIFKQPFMRPVQTTQEEDGCSCRFPEEEEGGCELRVRFSR | |
| | SADAPAYQQGQNQLYNELNLGRREEYDVLDRRRGRDPEMGGRPQRRRNPQ | |
| | EGLYNELQRDRMAEAYSEIGMRGERRRGRGHDGLYQGLSTATRDTYDALH | |
| | MQALPPR | |

*Described in U.S. 2011/0318360 A1

For both the α-PS CAR1 and α-PS CAR2 exemplary constructs, DNA sequences were optimized based on the preferred synonymous codons in human species. The full DNA polynucleotide sequences for the α-PS CAR1 and α-PS CAR2 constructs are shown below in Table 3 as SEQ ID NO: 11 and SEQ ID NO: 12, respectively.

TABLE 3
α-PS CAR1 and CAR2 DNA Polynucleotide Sequences

| Sequence | | SEQ |
|---|---|---|
| Construct | Polynucleotide Sequence | ID NO |
| Full α-PS CAR1 | ATGCATCCTCAAGTGGTCATCTTAAGCCTCATCCTACATCTGGCAGATTC | 11 |
| DNA polynucleotide | TGTAGCTGGTTACCCATACGATGTTCCAGATTACGCTTCTGTAAAGGTTG | |
| sequence | GTGGAGAGGCAGGTCCATCTGTCACACTACCCTGCCACTACAGTGGAGCT | |
| | GTCACATCCATGTGCTGGAATAGAGGCTCATGTTCTCTATTCACATGCCA | |
| | AAATGGCATTGTCTGGACCAATGGAACCCACGTCACCTATCGGAAGGACA | |
| | CACGCTATAAGCTATTGGGGGACCTTTCAAGAAGGGATGTCTCTTTGACC | |
| | ATAGAAAATACAGCTGTGTCTGACAGTGGCGTATATTGTTGCCGTGTTGA | |
| | GCACCGTGGGTGGTTCAATGACATGAAAATCACCGTATCATTGGAGATTG | |
| | TGCCACCCAAGGTCACGACTACTCCAATTGTCACAACTGTTCCAACCGTC | |
| | ACGACTGTTCGAACGAGCACCACTGTGCCCACAACCACTACAGTACCTAT | |
| | GACGACTGTTCCAACGACAACTGTTCCAACAACAATGAGCATTCCAACGA | |
| | CAACGACTGTTCTGACGACAATGACTGTTTCAACGACAACGAGCGTTCCA | |
| | ACGACAACGAGCATTCCAACAACAACAAGTGTTCCAGTGACAACAACTGT | |
| | CTCTACCTTTGTTCCTCCAATGCCTTTGCCCAGGCAGAACCATGAACCAG | |
| | TAGCCACTTCACCATCTTCACCTCAGCCAGCAGAAACCCACCCTACGACA | |
| | CTGCAGGGAGCAATAAGGAGAGAACCCACCAGCTCACCATTGTACTCTTA | |
| | CACAACAGATGGGAATGACACCGTGACAGAGTCTTCAGATGGCCTTTGGA | |
| | ATAACAATCAAACTCAACTGTTCCTAGAACATAGTCTACTGACGGCCAAT | |
| | ACCACTAAAGGAATCTATGCTGGAGTCTGTATTTCTGTCTTGGTGCTTCT | |
| | TGCTCTTTTGGGTGTCATCATTGCCAGAGTGAGATTCAGCAGGAGCGCAG | |
| | ACGCCCCCGCGTACCAGCAGGGCCAGAACCAGCTCTATAACGAGCTCAAT | |
| | CTAGGACGAAGAGAGGAGTACGATGTTTTGGACAGAAGACGTGGCCGGGA | |
| | CCCTGAGATGGGGGGAAGGCCGCAGAGAAGGAGAAACCCTCAGGAAGGCC | |
| | TGTACAATGAACTGCAGAGAGATAGGATGGCGGAGGCCTACAGTGAGATT | |
| | GGGATGAGAGGCGAGCGCCGGAGGGGCAGGGGGCACGATGGCCTTTACCA | |
| | GGGTCTCAGTACAGCCACCAGGGACACCTACGACGCCCTTCACATGCAGG | |
| | CCCTGCCCCCTCGCTAA | |
| Full α-PS CAR2 | ATGGCGTTACCTGTTACAGCACTCCTTTTGCCGCTCGCACTTCTTTTGCA | 12 |
| DNA polynucleotide | TGCAGCTAGACCTTATCCATATGATGTCCCAGATTATGCAAGCAGCGAGC | |
| sequence | TGAGCCAGGACCCCGCCGTGAGCGTGGCCCTGGGCCAGACCGTGAGGATC | |
| | ACCTGCCAGGGCGACAGCCTGAGGAGCTACTACGCCAGCTGGTACCAGCA | |
| | GAAGCCCGGCCAGGCCCCTGTGCTGGTGATCTACGGCAAGAACAACAGGC | |
| | CCAGCGGCATCCCCGACAGGTTCAGCGGCAGCAGCAGCGGCAACACCGCC | |
| | AGCCTGACCATCACCGGCGCCCAGGCCGAGGACGAGGCCGACTACTACTG | |
| | CAACAGCAGGGACAGCAGCGGCAACGTGGTGTTCGGCGGCGGCACCAAGG | |
| | TGACCGTGCTGGGTGGTGGTGGATCCGGCGGTGGTGGATCAGGCGGAGGA | |
| | GGTAGTGGAGGAGGTGGATCACAGGTGCAGCTGCAGGAGAGCGGCCCCGG | |
| | CCTGGTGAAGCCCAGCGGCACCCTGAGCCTGACCTGCGCCGTGAGCGGCG | |
| | GCAGCATCAGCAGCAGCAACTGGTGGAGCTGGGTGAGGCAGCCTCCCGGC | |
| | AAGGGCCTGGAGTGGATCGGCGAGATCTACCACAGCGGCAGCACCAACTA | |
| | CAACCCCAGCCTGAAGAGCAGGGTGACCATCAGCGTGGACAAGAGCAAGA | |
| | ACCAGTTCAGCCTGAAGCTGAGCAGCGTGACCGCCGCCGACACCGCCGTG | |
| | TACTACTGCGCCAGGAGCAGGTTCAGGAGCTGGCTGGTGAAGAGGAGGTA | |
| | CGTGTACTTCGACTACTGGGGCCAGGGCACCCTGGTGACCGTGAGCAGCA | |
| | CCACCACACCCGCCCCTAGGCCTCCTACCCCTGCTCCTACCATCGCCAGC | |
| | CAGCCACTTTCTTTGCGGCCAGAAGCATGTCGTCCTGCTGCTGGTGGAGC | |
| | AGTGCACACCAGGGGCCTGGACTTCGCCTGCGACATCTACATCTGGGCCC | |
| | CTCTGGCCGGCACCTGCGGCGTGCTGCTGCTGAGCCTGGTGATCACCCTG | |
| | TACTGCAAACGGGGCAGAAAGAAACTCCTGTATATATTCAAACAACCATT | |
| | TATGAGACCAGTACAAACTACTCAAGAGGAAGATGGCTGTAGCTGCCGAT | |
| | TTCCAGAAGAAGAAGAAGGAGGATGTGAACTGAGAGTGAGATTCAGCAGG | |
| | AGCGCAGACGCCCCCGCGTACCAGCAGGGCCAGAACCAGCTCTATAACGA | |
| | GCTCAATCTAGGACGAAGAGAGGAGTACGATGTTTTGGACAGAAGACGTG | |
| | GCCGGGACCCTGAGATGGGGGGAAGGCCGCAGAGAAGGAGAAACCCTCAG | |
| | GAAGGCCTGTACAATGAACTGCAGAGAGATAGGATGGCGGAGGCCTACAG | |
| | TGAGATTGGGATGAGAGGCGAGCGCCGGAGGGGCAGGGGGCACGATGGCC | |
| | TTTACCAGGGTCTCAGTACAGCCACCAGGGACACCTACGACGCCCTTCAC | |
| | ATGCAGGCCCTGCCCCCTCGCTAA | |

A negative control CAR construct having no extracellular domain region (no ECD) was also developed and used for experiments. This negative control construct lacks an extracellular antigen recognition domain and comprises the following sequence elements: the signal peptide from the CD8A protein (UniProt ID: P01732; amino acids 1-21), shown above as SEQ ID NO: 6 in Table 2; an HA tag, shown above as SEQ ID NO: 2 in Table 2; the hinge domain and the transmembrane domain from the CD8A protein (UniProt ID: P01732; amino acids 138-182), shown above as SEQ ID NO: 8 in Table 2; and the modified intracellular domain from the CD3Z protein (UniProt ID: P20963; amino acids 52-164) having residues 54, 88, 99, 104, 116, 118, 129, 136, and 150 mutated from a lysine to an arginine, shown above as SEQ ID NO: 4 in Table 2. An empty GFP vector was also used as a negative control in certain experiments.

KRAS inhibition (KRASi) was found to induce PS externalization in KRAS-addicted pancreatic cancer cells as indicated by Annexin V staining (FIG. 1B). The α-PS CAR1 construct was found to significantly increase human THP-1 differentiated macrophage phagocytic activity about three-fold compared to the negative GFP control against KRASi-treated human cancer cells versus untreated cancer cells (FIG. 1C). The cytotoxic killing activity of macrophages against KRASi-treated cancer cells was also improved with the α-PS CAR2 construct (FIG. 1D). Moreover, inhibition of the negative signal input through the merTK pathway synergistically enhanced cancer cell killing by α-PS CAR2 macrophages in a dose dependent manner (FIG. 1D).

### Example 2

### Virus Production

To overexpress the α-PS CAR constructs in the human monocytic cell line THP-1, in primary monocytic cells from human peripheral blood or mouse bone marrow, and in human induced pluripotent stem cells (iPSCs), lentiviral vectors such as pHAGE and pLenti were used. The lentiviral vectors constitutively express GFP, so that infection efficiency may be determined by measuring GFP+ cells via flow cytometry. Second- or third-generation lentiviral vector packaging systems were used for the study. Briefly, for the second-generation lentiviral packaging system, pMd2G, pPAX2, and expression plasmid were required for producing lentivirus. For the third-generation lentiviral packaging system, pRSV-Rev, pVSVG, pMDLg/pRRE, and expression plasmid were required for producing lentivirus.

THP-1 monocytic cells or human iPSCs were infected by lentivirus expressing the α-PS CAR constructs, followed by cell sorting to enrich and identify the GFP+ cell population. The cell surface expression of the α-PS CAR was examined by staining for the HA tag, which was added near the N-terminus of the α-PS CAR constructs.

### Example 3

### Effector Cells and Target Cells

The monocytic cell line THP-1, human iPSCs (BYS0110 and BXS0116), human pancreatic ductal adenocarcinoma (PDAC) cell lines (AsPC-1 and MIA PaCa-2), and human non-small cell lung cancer (NSCLC) cell lines (NCI-H23 and NCI-H1373) were from the American Type Culture Collection (ATCC) and were cultured following the manufacturer's instructions. In addition, mouse PDAC cell lines derived from KPC (KRASG12D L/+, P53 L/+, p48-Cre+) genetically engineered mice were also examined. The cancer cell lines expressed a luciferase reporter and a red fluorescent protein (RFP) for functional assays.

### Example 4

### Phagocytosis Assays

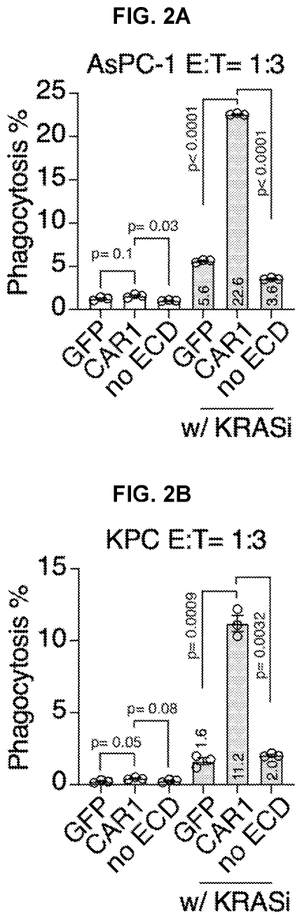

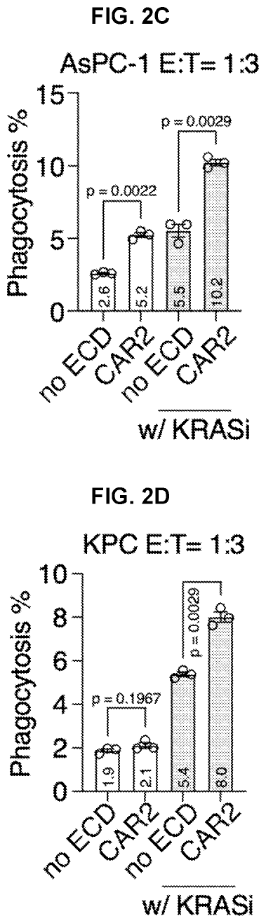

To determine the level of phagocytic activity by macrophages expressing the α-PS CAR constructs (i.e., α-PS CAR-Ms), the α-PS CAR-Ms and cancer cells were co-cultured at an effector cell:target cell (E:T) ratio of 1:3, as indicated in FIG. 2A-D. Cancer cells were treated with the α-PS CAR-M with or without an additional targeted therapy regimen (e.g., mutant KRAS inhibitors (KRASi)) to increase PS externalization. The KRAS G12D inhibitor MRTX1133 was used for these experiments. Flow cytometry analysis or imaging was performed after co-culture for 1-4 hours. The phagocytic index (phagocytosis %) was calculated as the percentage of RFP+GFP+ double-positive cells in GFP+ CAR-Ms. The phagocytic activities of the α-PS CAR-Ms are shown in FIG. 2A-D as phagocytosis percentage. Unpaired two-tailed t tests were used for statistical analysis.

### Example 5

### Killing Assays

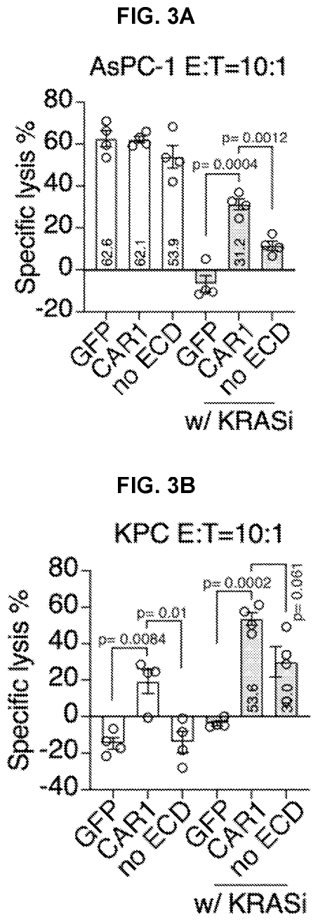

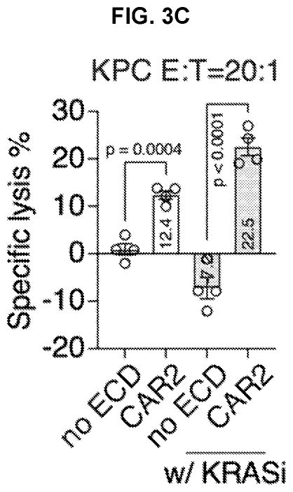

To determine the level of antigen-specific killing by the α-PS CAR-Ms, the α-PS CAR-Ms and cancer cells were co-cultured at an E:T ratio of 10:1 or 20:1, as indicated in FIG. 3A-C. Cancer cells were treated with the α-PS CAR-M with or without an additional targeted therapy regimen (e.g., KRASi) to increase PS externalization. The KRAS G12D inhibitor MRTX1133 was used for these experiments. Luciferase activity was measured in residue cancer cells by bioluminescent assay following co-culture for 24 hours. The specific lysis percentage was calculated as: (sample signal−tumor alone)/(background signal−tumor alone)×100. The specific cytolytic activities of the α-PS CAR-Ms are shown in FIG. 3A-C as specific lysis percentage. Unpaired two-tailed t tests were used for statistical analysis.

### Example 6

### Live-Cell Phagocytosis Imaging Assays

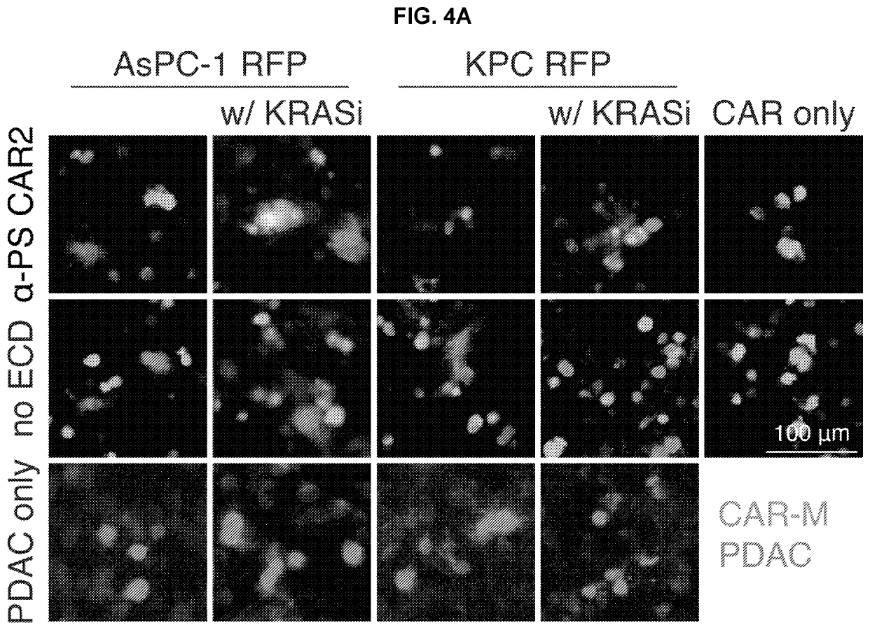

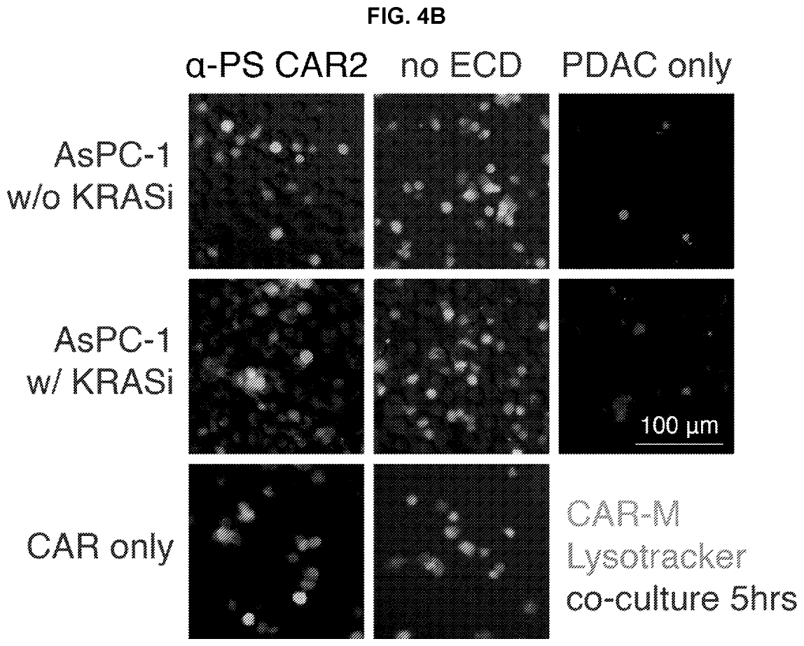

Live-cell imaging analysis was performed to assess the phagocytosis activity of α-PS CAR-Ms co-cultured with cancer cells for 5 hours. FIG. 4A-B show fluorescent images of phagocytosis and autophagy using the IncuCyte® live-cell imaging analysis system. FIG. 4A shows phagocytosis imaging of α-PS CAR2-Ms labeled by GFP (green) and co-cultured AsPC-1 PDAC cells or mouse KPC PDAC cells labeled by CellTracker® Red (red; red fluorescent protein (RFP)), where the co-staining (yellow) indicated phagocytic events. High phagocytic activities were observed in the co-cultures of α-PS CAR2-Ms with KRASi-treated cancer cells as compared to cancer cells not treated with KRASi or to co-cultures with the control CAR-Ms lacking an extracellular domain (no ECD) (FIG. 4A).

In addition, active phagocytosis is known to be accompanied by increased autophagy. FIG. 4B shows autophagy activity of α-PS CAR2-Ms labeled by GFP (green). LysoTracker® Red (red) was used to visualize autophagy activities. The co-staining (yellow) indicated phagocytic events. The LysoTracker® assay revealed higher autophagy activity in the co-cultures of α-PS CAR2-Ms with KRASi-treated AsPC-1 cancer cells as compared to cancer cells not treated with KRASi or to co-cultures with the control CAR-Ms lacking an extracellular domain (no ECD) (FIG. 4B).

### Example 7

### In Vivo Assessment of α-PS CAR-Ms

The tumoricidal potential of the α-PS CAR macrophage therapy described herein is investigated using in vivo models, such as rodent models having solid tumor cancers. Phosphatidylserine (PS) is an amino-phospholipid having a structure that is conserved in humans and mice. Therefore, pre-clinical in vivo studies using both human xenograft mouse models and immune competent mice with spontaneous tumors are performed to assess the effectiveness of the α-PS CAR macrophages.

1. A polynucleotide construct encoding a phosphatidylserine (PS)-targeted chimeric antigen receptor (CAR) polypeptide, the PS-targeted CAR polypeptide comprising:
(a) a signal peptide;
(b) an extracellular PS-binding domain;
(c) a hinge domain;
(d) a transmembrane domain; and
(e) one or more intracellular immunostimulatory activation domains.

2. The polynucleotide construct of claim 1, wherein the signal peptide comprises an amino acid sequence having at least 90-99% identity to SEQ ID NO: 1 or 6.

3. The polynucleotide construct of claim 1, wherein the extracellular PS-binding domain comprises an amino acid sequence having at least 90-99% identity to one or more portions of SEQ ID NO: 3 or 7.

4. The polynucleotide construct of claim 1, wherein the hinge domain comprises an amino acid sequence having at least 90-99% identity to one or more portions of SEQ ID NO: 3 or 8.

5. The polynucleotide construct of claim 1, wherein the transmembrane domain comprises an amino acid sequence having at least 90-99% identity to one or more portions of SEQ ID NO: 3 or 8.

6. The polynucleotide construct of claim 1, wherein the one or more intracellular immunostimulatory activation domains comprise one or more amino acid sequences having at least 90-99% identity to SEQ ID NO: 4 or 9.

7. The polynucleotide construct of claim 1, wherein the PS-targeted CAR polypeptide comprises an amino acid sequence having at least 90-99% identity to SEQ ID NO: 5 or 10.

8. The polynucleotide construct of claim 7, wherein the PS-targeted CAR polypeptide comprises an amino acid sequence of SEQ ID NO: 5 or 10.

9. The polynucleotide construct of claim 1, wherein the polynucleotide construct comprises a polynucleotide sequence having at least 90-99% identity to SEQ ID NO: 11 or 12.

10. The polynucleotide construct of claim 9, wherein the polynucleotide construct comprises a polynucleotide sequence of SEQ ID NO: 11 or 12.

11. A vector comprising the polynucleotide construct of claim 1, the vector comprising a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof.

12. The vector of claim 11, wherein the vector is a lentiviral vector selected from the group consisting of pHAGE and pLenti.

13. A phosphatidylserine (PS)-targeted chimeric antigen receptor (CAR) polypeptide encoded by the polynucleotide construct of claim 1.

14. A modified macrophage or monocyte comprising the polynucleotide construct or PS-targeted CAR polypeptide of claim 1.

15. A modified macrophage or monocyte comprising a surface-expressed phosphatidylserine (PS)-targeted chimeric antigen receptor (CAR) polypeptide, the PS-targeted CAR polypeptide comprising:
(a) a signal peptide;
(b) an extracellular PS-binding domain;
(c) a hinge domain;
(d) a transmembrane domain; and
(e) one or more intracellular immunostimulatory activation domains.

16. The modified macrophage or monocyte of claim 15, wherein the modified macrophage or monocyte is derived from a primary macrophage or monocyte, or wherein the modified macrophage or monocyte is derived from an induced pluripotent stem cell.

17. The modified macrophage or monocyte of claim 15, wherein the PS-targeted CAR polypeptide comprises an amino acid sequence having at least 90-99% identity to SEQ ID NO: 5 or 10, or has the amino acid sequence of SEQ ID NO: 5 or 10.

18. (canceled)

19. The modified macrophage or monocyte of claim 15, wherein the PS-targeted CAR polypeptide is encoded by a polynucleotide construct comprising a polynucleotide sequence having at least 90-99% identity to SEQ ID NO: 11 or 12, or is encoded by a Polynucleotide construct comprising a polynucleotide sequence of SEQ ID NO: 11 or 12.

20. (canceled)

21. A pharmaceutical composition comprising the modified macrophage or monocyte of claim 15.

22. A method of treating cancer in a subject, the method comprising administering to the subject a therapeutically effective amount of a pharmaceutical composition comprising a modified macrophage or monocyte comprising a surface-expressed phosphatidylserine (PS)-targeted chimeric antigen receptor (CAR) polypeptide, the PS-targeted CAR polypeptide comprising:
(a) a signal peptide;
(b) an extracellular PS-binding domain;
(c) a hinge domain;
(d) a transmembrane domain; and
(e) one or more intracellular immunostimulatory activation domains.

23-26. (canceled)


SEQUENCE LISTING (12 sequences)

SEQ ID NO: 1 - protein, 20 aa, Homo sapiens
MHPQVVILSLILHLADSVAG

SEQ ID NO: 2 - protein, 9 aa, synthetic construct
YPYDVPDYA

SEQ ID NO: 3 - protein, 296 aa, Homo sapiens
SVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIA

SEQ ID NO: 4 - protein, 113 aa, synthetic construct
RVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 5 - protein, 438 aa, synthetic construct
MHPQVVILSLILHLADSVAGYPYDVPDYASVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIARVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 6 - protein, 21 aa, Homo sapiens
MALPVTALLLPLALLLHAARP

SEQ ID NO: 7 - protein, 277 aa, synthetic construct
SSELSQDPAVSVALGQTVRITCQGDSLRSYYASWYQQKPGQAPVLVIYGKNNRPSGIPDRFSGSSSGNTASLTITGAQAEDEADYYCNSRDSSGNVVFGGGTKVTVLGGGGSGGGGSGGGGSGGGGSQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARSRFRSWLVKRRYVYFDYWGQGTLVTVSSTTTPAPRPPTPAPTIASQPLSLRP

SEQ ID NO: 8 - protein, 45 aa, Homo sapiens
EACRPAAGGAVHTRGLDFACDIYIWAPLAGTCGVLLLSLVITLYC

SEQ ID NO: 9 - protein, 42 aa, Homo sapiens
KRGRKKLLYIFKQPFMRPVQTTQEEDGCSCRFPEEEEGGCEL

SEQ ID NO: 10 - protein, 507 aa, synthetic construct
MALPVTALLLPLALLLHAARPYPYDVPDYASSELSQDPAVSVALGQTVRITCQGDSLRSYYASWYQQKPGQAPVLVIYGKNNRPSGIPDRFSGSSSGNTASLTITGAQAEDEADYYCNSRDSSGNVVFGGGTKVTVLGGGGSGGGGSGGGGSGGGGSQVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARSRFRSWLVKRRYVYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVRFSRSADAPAYQQGQNQLYNELNLGRREEYDVLDRRRGRDPEMGGRPQRRRNPQEGLYNELQRDRMAEAYSEIGMRGERRRGRGHDGLYQGLSTATRDTYDALHMQALPPR

SEQ ID NO: 11 - DNA, 1317 bp, synthetic construct
  source 1..1317: mol_type = other DNA
atgcatcctcaagtggtcatcttaagcctcatcctacatctggcagattctgtagctggttacccatacgatgttccagattacgcttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgtgcccacaaccactacagtacctatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccagagtgagattcagcaggagcgcagacgcccccgcgtaccagcagggccagaaccagctctataacgagctcaatctaggacgaagagaggagtacgatgttttggacagaagacgtggccgggaccctgagatggggggaaggccgcagagaaggagaaaccctcaggaaggcctgtacaatgaactgcagagagataggatggcggaggcctacagtgagattgggatgagaggcgagcgccggaggggcagggggcacgatggcctttaccagggtctcagtacagccaccagggacacctacgacgcccttcacatgcaggccctgccccctcgctaa

SEQ ID NO: 12 - DNA, 1524 bp, synthetic construct
  source 1..1524: mol_type = other DNA
atggcgttacctgttacagcactccttttgccgctcgcacttcttttgcatgcagctagaccttatccatatgatgtcccagattatgcaagcagcgagctgagccaggaccccgccgtgagcgtggccctgggccagaccgtgaggatcacctgccagggcgacagcctgaggagctactacgccagctggtaccagcagaagcccggccaggcccctgtgctggtgatctacggcaagaacaacaggcccagcggcatccccgacaggttcagcggcagcagcagcggcaacaccgccagcctgaccatcaccggcgcccaggccgaggacgaggccgactactactgcaacagcagggacagcagcggcaacgtggtgttcggcggcggcaccaaggtgaccgtgctgggtggtggtggatccggcggtggtggatcaggcggaggaggtagtggaggaggtggatcacaggtgcagctgcaggagagcggccccggcctggtgaagcccagcggcaccctgagcctgacctgcgccgtgagcggcggcagcatcagcagcagcaactggtggagctgggtgaggcagcctcccggcaagggcctggagtggatcggcgagatctaccacagcggcagcaccaactacaaccccagcctgaagagcagggtgaccatcagcgtggacaagagcaagaaccagttcagcctgaagctgagcagcgtgaccgccgccgacaccgccgtgtactactgcgccaggagcaggttcaggagctggctggtgaagaggaggtacgtgtacttcgactactggggccagggcaccctggtgaccgtgagcagcaccaccacacccgcccctaggcctcctacccctgctcctaccatcgccagccagccactttctttgcggccagaagcatgtcgtcctgctgctggtggagcagtgcacaccaggggcctggacttcgcctgcgacatctacatctgggcccctctggccggcacctgcggcgtgctgctgctgagcctggtgatcaccctgtactgcaaacggggcagaaagaaactcctgtatatattcaaacaaccatttatgagaccagtacaaactactcaagaggaagatggctgtagctgccgatttccagaagaagaagaaggaggatgtgaactgagagtgagattcagcaggagcgcagacgcccccgcgtaccagcagggccagaaccagctctataacgagctcaatctaggacgaagagaggagtacgatgttttggacagaagacgtggccgggaccctgagatggggggaaggccgcagagaaggagaaaccctcaggaaggcctgtacaatgaactgcagagagataggatggcggaggcctacagtgagattgggatgagaggcgagcgccggaggggcagggggcacgatggcctttaccagggtctcagtacagccaccagggacacctacgacgcccttcacatgcaggccctgccccctcgctaa